(19) United States
(12) Patent Application Publication
(10) Pub. No.: US 2003/0157208 A1
(43) Pub. Date: Aug. 21, 2003

(54) Cynomoriaceae extract and use in treating erectile dysfunction

(76) Inventors: Rui Wang, Saskatoon (CA); Yaoge Huang, Saskatoon (CA); Weimin Zhao, Nepean (CA)

Correspondence Address: GREENLEE WINNER AND SULLIVAN P C, 5370 MANHATTAN CIRCLE, SUITE 201, BOULDER, CO, 80303, US

(21) Appl. No.: 10/327,624

(22) Filed: Dec. 20, 2002

Related U.S. Application Data
(60) Provisional application No. 60/341,210, filed on Dec. 20, 2001.

(51) Int. Cl. A61K 35/78

(52) U.S. Cl. 424/779

(57) ABSTRACT

An extract from the herb of the family Cynomoriaceae, preferably Cynomorium songaricum Rupr, which is also commonly known as Suo Yang in Chinese Medicine, has been prepared and found to have medicinal properties. The extract is preferably prepared from stems of the plant, and is most preferably extracted with an solvent including butanol, herein abbreviated as “BuSY”. The extract is shown to have efficacy in the treatment of erectile dysfunction and the enhancement of male sexual health in vertebrates. A pharmaceutical preparation of this extract can be formulated with a pharmaceutically acceptable carrier to treat erectile dysfunction in host animals. The components of the nutraceutical extract can be combined in differing combinations to enhance and/or improve male sexual health.

### CROSS REFERENCE TO RELATED APPLICATION

This application claims priority from U.S. Provisional Patent Application No. 60/341,210, filed Dec. 20, 2001. To the extent that it is consistent herewith, the aforementioned application is incorporated herein by reference.

### FIELD OF THE INVENTION

The present invention relates to a plant extract prepared from the family of Cynomoriaceae such as Cynomorium songaricum Rupr. and its use in the treatment of erectile dysfunction or impotence in host animals.

### BACKGROUND OF THE INVENTION

Erectile dysfunction or ED (also called impotence) affects over 30 million men, about half of all men aged between 40 to 70 years, to some degree in the United States, and much more in the world. ED is the inability for a man to get and/or keep an erection sufficient for sexual activity. When a man is sexually aroused, the arteries in the penis relax and widen, allowing more blood to flow into the penis. As the arteries in the penis expand and harden, the veins that normally carry blood away from the penis become compressed, restricting the blood flow out of the penis. With more blood flowing in and less flowing out, the penis enlarges, resulting in an erection. In men with ED, the chemical reactions responsible for erections do not take place as usual, so the arteries don't expand and the penis cannot fill with blood. The majority of cases of ED are associated with physical conditions, not just age. ED is a treatable condition in most men who have it.

Current treatment protocols include the administration (through prescription) of sildenafil. VIAGRA® is the citrate salt of sildenafil, made by Pfizer, Inc. VIAGRA® is a selective inhibitor of cyclic guanosinemonophosphate (cGMP)-specific phosphodiesterase type 5 (PDE5). VIAGRA® received US FDA approval in 1998. It is available in tablet form with the equivalent of 25 mg, 50 mg and 100 mg of sildenafil or oral administration. In addition to the active ingredient, sildenafil citrate, each tablet contains the following inactive ingredients: microcrystalline cellulose, anhydrous dibasic calcium phosphate, croscarmellose sodium, magnesium stearate, hydroxypropyl methylcellulose, titanium dioxide, lactose, triacetin, and FD & C Blue #2 aluminum lake. This composition is known to have several side effects and is not effective in treating ED in all male patients. Many pharmaceutical companies are actively developing compounds which may compete with sildenafil and have lesser side effects. A natural and organic alternative treatment with the same or similar benefits to VIAGRA® would be advantageous.

Traditional Chinese Medicine (TCN) indicates that Cynomorium, along with many other plants or herbs, have activity to “invigorate the kidney and supplement essence”, that is for deficiency of kidney-yang and insufficiency of essence and blood manifested as impotence, emission, flaccidity or paralysis of extremities, cold pain of the loin and knees (see Ou Ming, 1992). However, to date little scientific evidence of utility, or extraction protocol exists.

### SUMMARY OF THE INVENTION

The present invention provides a novel extract of Cynomorium obtained by solvent extraction from the stems of a plant of the family Cynomoriaceae. Preferred plant sources of the extract are Cynomorium songaricum Rupr. and Cynomorium coccineum L, with Cynomorium songaricum Rupr. being the most preferred source. The extract is preferably prepared from the stem of the plant, with the most preferred solvent including butanol.

The invention also provides a pharmaceutical composition comprising an extract of Cynomorium as set out above, in admixture with one or more pharmaceutically acceptable carriers.

The invention also provides a method of treating erectile dysfunction by administering a therapeutically effective amount of an extract of Cynomorium as set forth above to a host animal.

The invention also extends to the use of an extract of Cynomorium as set forth above, for the preparation of a pharmaceutical composition for the treatment of erectile dysfunction.

The invention also extends to a method of preparing an extract of Cynomorium, comprising contacting a powder or pulp obtained from the plant or plant parts of the family Cynomoriaceae with one or more organic or aqueous extraction solvents to remove an extract; and isolating the extract with activity for alleviating or preventing symptoms of erectile dysfunction. Most preferred solvents and purification techniques are disclosed herein.

This extracts of this invention are shown to be effective in the treatment of ED in an animal model system. Additionally, since the extract is prepared from a natural, edible products the potential for side effects is decreased.

As used herein and in the claims, the terms and phrases set out below have the meanings which follow.

“Active ingredient” means any extract or composition thereof capable of modifying or modulating the function of at least one given biological system.

“Erectile dysfunction” means the inability to achieve and/or maintain an erection sufficient for sexual activity.

“Biocompatible” means generating no significant undesirable host response for the intended utility. Most preferably, biocompatible materials are non-toxic for the intended utility. Thus, for human utility, biocompatible is most preferably non-toxic to humans or human tissues.

“Cynomorium” is meant to refer to at least one plant of the family Cynomoriaceae, including, but not limited to Cynomorium songaricum Rupr., Cynomorium coccineum L., or other species of Cynomorium.

“Order” means a taxonomic category of related organisms with a category consisting of a number of similar families.

“Family” means a taxonomic category of related organisms ranking below the order and above the genus.

“Species” means a taxonomic category ranking below a genus and consisting of a group of closely related individuals.

“Extract” means a crude extract, purified extract, and purified composition obtained by solvent extraction, with or without purification of the extract, from a plant or parts thereof of Cynomorium.

“Purified” means partially purified and/or completely purified. Thus, a “purified composition” may be either partially purified or completely purified.

“Plant or parts thereof” means either the whole plant, or any part of the plant such as an aerial part, fruit, leaf, stem, or root and any combination thereof.

“Pharmaceutically- or therapeutically- or nutraceutically-acceptable” is used herein to denote a substance which does not significantly interfere with the effectiveness or the biological activity of the active ingredient (erectile dsyfunction activity) and which has an acceptable toxic profile for the host to which it is administered.

“Therapeutically or nutraceutically effective amount” is used herein to denote any amount of a formulation of the extract which will exhibit a positive activity for alleviating or preventing symptoms of erectile dysfunction upon administration. The amount of extract administered will vary with the condition being treated, the stage of advancement of the condition, the age and type of host, and the type and concentration of the formulation being applied. Appropriate amounts in any given instance will be readily apparent to those skilled in the art or capable of determination by routine experimentation.

“Carrier” means a suitable vehicle which is biocompatible and pharmaceutically acceptable, including for instance, one or more solid, semisolid or liquid diluents, excipients, adjuvants, flavours, or encapsulating substances which are suitable for administration.

“Host” or “host animal” means humans or other invertebrates.

The butanol fraction of Cynomorium songaricum Rupr. extract is herein abbreviated as “BuSY”.

### BRIEF DESCRIPTION OF THE DRAWINGS

FIG. 1 is a flow diagram which illustrates preferred extraction steps for preparing the extract from Suo Yang (Cynomorium songaricum Rupr).;

FIG. 2 is a schematic illustration of a thin layer chromatographic (TLC) separation as a purification technique of BuSY;

FIG. 3A and 3B are schematic illustrations of the anatomical location and dissection of rat corpus cavernosum tissue for testing the effect BuSY on rat penis tissue, with FIG. 3A showing the portion of the penis which is tested, and FIG. 3B showing a transversal cut of that test portion of the penis;

FIG. 4 is a schematic illustration of the experimental apparatus used to measure the contractility or relaxation of the penis tissue with the BuSY extracts;

FIG. 5 is a graph depicting the comparison of relaxation events in penis tissue between BuSY and sildenafil (VIAGRA®); and

FIG. 6 is a graph showing a comparison of the relaxation potencies in norepinephrine precontracted penile tissue between BuSY608 (one component purified from BuSY using TLC) and sildenafil (VIAGRA®).

### DESCRIPTION OF THE PREFERRED EMBODIMENTS

i. Preparation and Purification of Cynomorium Extracts

The extract of the present invention is prepared from plants in the family of Cynomoriaceae, most preferably from Cynomorium songaricum Rupr. Other species of Cynomoriaceae may be used, for instance Cynomorium coccineum L. The common herbal name for Cynomorium songaricum Rupr. is Herba Cynomorii. Chinese Names for this herb include: Suo Yang, Bu Lao Cao, Xiu Tie Bang, Di Mao Qiu, Huang Gu Lang, and Suo Yan Zi. While the whole plant or parts thereof may be used, the most useful plant part for the extracts of the present invention is the fleshy stem of the plant. Characteristics of this plant include: Stem slightly curved and flat-cylinder, 5-15 cm long, 1.5-5.0 cm in diameter; surface brown, coarse, or with triangular scale leaves remaining and prominent longitudinal grooves. The stems are prepared by crosscutting, with sections showing triangular vascular bundles.

In order to prepare extracts of Cynomorium, plants or parts thereof may be provided as a powder (commercially available), or may be crushed and ground from a dry form of the plant or plant parts to obtain a powder, or the plants or plant parts may be masticated to form a plant pulp or mash which can, if desired, be dried and ground. The powder or pulp can then be extracted with one or more organic and/or aqueous extraction solvents. The solvent is then removed from the extract. The whole plant may be used or parts of the plant. Most preferably, the stem of the plant is used. Preferably, the extract is then purified to yield a purified extract or one or more purified compositions. Exemplary purification and isolation techniques include column chromatography and fractional distillation.

The extraction process of the present invention is desirably carried out using an organic or aqueous extraction solvent or a mixture of organic and/or aqueous extraction solvents. While butanol is the most preferred solvent used in the extraction process, other single solvents or solvent mixtures may be used. Preferred solvents have a dielectric constant (specific inductive capacity) of the solvent(s) in the range of ε=6.11 to 31.2 (see Bejing, 1980). Exemplary solvents include, alone or in admixture, butanol, methanol, ethanol, propanol, isobutyl alcohol, pentanol, isoamyl alcohol, hexanol, hexanediol, acetone, ethyl acetate, water and similar solvents. To assist in separating the extract of the present invention into the organic or aqueous extraction solvent(s), other solvents with lower dielectric constants may be used for the discard fractions such as hexane, benzene, petroleum ether, chloroform and ethyl acetate.

ii. Determination of Activity for Alleviating or Preventing Symptoms of Erectile Dysfunction

The extracts of this invention may be tested for their activity for alleviating or preventing symptoms erectile dysfunction by techniques known in the art. The test is similar to the endothelium-dependent vasorelaxatin test reported by Fitzpatrick et al., 1995. In general, in accordance with this protocol, plant extracts are tested for endothelium-dependent relaxation (EDR) by measuring the relaxation or contraction imparted by diluted samples to an appropriate tissue sample precontacted by norepinephrine to arrive at an EDR value that is the percentage of relaxation imparted. Generally, an extract is determined to have substantial positive EDR activity, and thus a positive effect on alleviating or preventing the symptoms of erectile dysfunction, if it can induce a positive value EDR of 50% (ED50) or higher, however, extracts having a lower positive EDR value as low as 20% may be useful for alleviating or preventing symptoms of erectile dysfunction.

iii. Formulations, Formulating, Dosages, and Treatment

The extracts of the present invention may be formulated in any form, for example as powders, liquids, capsules, pills, caplets, capsules, drinks, tinctures, or injectable formulations (see Xi Nianzhu, 1997), and may be administered orally, intraperitoneally, or intravenously.

Powders of the extract may be used in that form directly as a loose powder or encapsulated powder. Alternatively, powders may be formulated into capsules, caplets, tablets and similar dosage forms. Further, powders may be formulated within liquid pervious membranes such as filters, meshes and the like, such as a tea bag-type infuser, for generating liquids containing the dissolved extract. The powder form of the extract may be incorporated into liquids, formulated as solutions, dispersions or suspensions by dissolving the extract, for example as a drink, tincture, or drop. The extract may be administered alone, or with a carrier such as saline solution, an alcohol or water. An effective daily amount of the extract will vary with the subject, but will be less than is toxic while still providing a therapeutic effect. Solutions and formulations of the extract may lose some activity with aging and are thus either prepared in stable forms, or preferably prepared fresh for administration, for example in multi-component kit form so as to avoid aging and to maximize the therapeutic effectiveness of the extract. Suitable kits or containers are well known for maintaining the phases of formulations separate until the time of use. A kit containing the extract in powder form may provide a sterile carrier such as water (and other ingredients) in a separate container in dosage specific amounts. The extract may be provided in a “tea bag”-type infuser or pouch, for generating liquid formulations at the time of use.

It will be understood that extracts and pharmaceutical compositions should be appropriately sterilized, purified and/or tested for microbiological parameters to ensure safety of administration. Extracts and pharmaceutical compositions should be sealed in appropriate packaging or containers which for example, limit moisture (as in the case of powders or encapsulated powders) which could impair the activity of the extract for its intended utility.

Typically, the extract will be formulated in one or more of the forms set out above. The extract can be prepared alone or as an active ingredient in pharmaceutical compositions including non-toxic, pharmaceutically acceptable carriers, diluents and excipients, as are well known, see for example Merck Index, and Gilman et al., (1996) Goodman and Gilman's: The Pharmacological Bases of Therapeutics. Exemplary as excipients are saline, glucose, starch etc. For standard dosages of conventional pharmacological agents, see, e.g., Physicians Desk Reference (1997 Edition); and U.S. Pharmacopeia National Formulary (1995). Compositions may also include flavours, colourings, coatings, etc. All agents must be non-toxic and physiologically acceptable for the intended purpose, and must not substantially interfere with the activity of the extract so as to deleteriously affect the effect on alleviating or preventing the symptoms of erectile dysfunction. Ingredients are thus only included in therapeutically acceptable amounts.

The dosage of the extract depends upon many factors that are well known to those skilled in the art, for example, the particular form of the extract; the stage of advancement of the condition, the age, weight and type of host, the type and concentration of the formulation being applied, the concurrent therapeutic treatments; and the experience and judgement of the clinician or practitioner administering the therapy. A therapeutically effective amount of the extract provides either subjective relief of symptoms or an objectively identifiable improvement as noted by the clinician or other qualified observer. The extract may be administered orally, intraperitoneally, or intravenously at a dosage range and frequency (e.g., at least once daily) such that the level of active extract is maintained in the body. The dosage range varies with the route of administration, and the form and potency of the extract; for example, one dose of the extract in a capsule taken orally may contain for example 0.001-30 g per day, preferably 0.5-20 g, more preferably 2-15 g, and most preferably 5-10 g (see Chen Qi, 1994).

The extracts of this invention may be employed either alone or in combination with other compounds as a part of combination therapy, partially or completely, in place of other ED therapies.

The extracts of the present invention may be employed for the treatment and/or prevention of ED in a number of host animals. Besides being useful for human treatment, these extracts are also useful for veterinary treatment of companion animals, exotic animals and farm animals, including mammals, rodents, avians, and the like. More preferred animals include cattle, horses, dogs, cats, sheep and pigs.

Abbreviations and nomenclature employed herein are standard in the art and are commonly used in scientific publications such as those cited herein.

The invention is further illustrated by the following non-limiting examples.

### EXAMPLE 1

### Preparation of the Butanol Fraction of Cynormorium songaricum Rupr. (Suo Yang) Extract

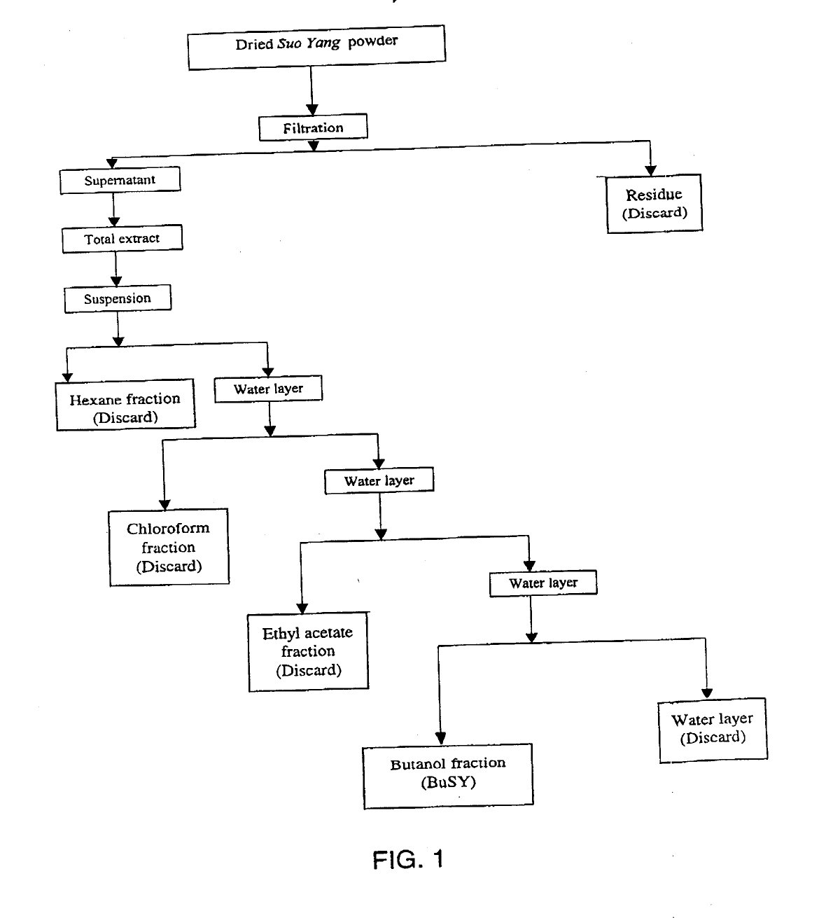

Extract Preparation (Flow chart is included in FIG. 1):

i. Stage 1—Total extraction

1. The slices of Herba Cynomorii were dried in an oven at 35° C. for 48 hrs.

2. The dry Herba Cynomorii was ground to powder, using a commercial blender at the setting 4 out of 1-4 range (ChampHP3, Model ES-3, K-TEC, Orem, Utah, USA).

3. The powder was soaked with 100% methanol at a ratio of 1 mg powder/5 ml methanol for 24 hrs at room temperature.

4. Filtration—The supernatant methanol solution which contains the total extract was poured through a filter paper (Grade 202, size 18.5 cm, Whatman Inc., Clifton, USA), and the precipitant powder was kept for the next methanol soaping cycle. This protocol was repeated 3 to 5 times, until the supernatant methanol solution became colourless.

5. The filtrate was evaporated by an evaporator (Buchi Rotavapor R-2000, CH-9230 Flawil, Switzerland) at 45° C. The extract was collected from the bottom of the evaporator flask. The yield of total methanol extract extracted from the raw Herba Cynomorii powder was 21.78% (by dry weight).

ii. Stage 2—The first water fraction

1. The total methanol extract was diluted with double distilled water at a ratio of 1 mg extract per 10 ml water. This diluted solution formed a suspension. The suspension was put in a separating funnel.

2. Hexane (100%) was added to the separating funnel at a ratio of 10 ml hexane/10 ml extract suspension. The mixture was stirred first and then kept unstirred until two layers separated with the hexane layer on the top and the water layer on the bottom.

3. The water layer was released from the separating funnel into another container, and was re-extracted with hexane as described above. This process was usually repeated for 5 to 6 times until no colour could be seen in the colourless hexane layer. The water layer collected after all these hexane extraction procedure was used in Stage 3 to extract a second water fraction using chloroform.

4. The top layer was put into an evaporator flask. Then, the hexane solution containing extract was evaporated by the evaporator as mentioned above at 35° C. The extract was collected from the bottom of the evaporator flask. The yield of hexane fraction was 9.30% (by dry weight of the total methanol extract).

iii. Stage 3—The second water fraction

1. The water layer collected after different steps of hexane extraction procedure was put into a separating funnel. Chloroform (100%) was added to this funnel at a ratio of 10 ml chloroform/10 ml water layer. The mixture was stirred first and then kept unstirred until two layers separated with the water layer on the top and the chloroform layer on the bottom.

2. The chloroform layer was evaporated by an evaporator at 35° C. and the yield of chloroform fraction separated from the total methanol extract was 0.55% (by dry weight).

3. The water layer left in the separating funnel was re-extracted with chloroform as described above. This process was usually repeated for 5 to 6 times until no colour could be seen in the colourless chloroform layer. The water layer collected after all these chloroform extraction procedure was used in Stage 4 to extract a third water fraction with ethyl acetate.

iv. Stage 4—The third water fraction

1. The water layer collected after different steps of chloroform extraction procedure was put into a separating funnel. Ethyl acetate (100%) was added to this funnel at a ratio of 10 ml ethyl acetate/10 ml water layer. The mixture was stirred first and then kept unstirred until two layers separated with the ethyl acetate layer on the top and the water layer on the bottom.

2. The water layer was released from the separating funnel into another container, and was re-extracted with ethyl acetate as described above. This process was usually repeated for 5 to 6 times until no colour could be seen in the colourless ethyl acetate layer. The water layer collected after all these ethyl acetate extraction procedure was used in Stage 5 to extract a final butanol fraction.

3. The ethyl acetate layer was evaporated by an evaporator at the temperature of 35° C. and the yield of ethyl acetate fraction separated from total methanol extract was 1.73% (by dry weight).

v. Stage 5—The butanol fraction

1. The water layer collected after different steps of ethyl acetate extraction procedure was put into a separating funnel. Butanol (100%) was added to this funnel at a ratio of 10 ml butanol per 10 ml water layer. The mixture was stirred first and then kept unstirred until two layers separated with the butanol layer on the top and the water layer on the bottom.

2. The water layer was released from the separating funnel into another container, and was re-extracted with butanol as described above. This process was usually repeated for 5 to 6 times until no colour could be seen in the colourless butanol layer. 3. The butanol layer was evaporated by an evaporator at 80° C. and the yield of butanol fraction separated from total methanol extract was 14.20% (by dry weight). 4. The yield of butanol fraction (BuSY) from raw Herba Cynomorii powder was 21.78%×14.20%=3.09%.

The above methodology describes a preferred embodiment. The extraction steps can be changed, but butanol is preferred as the major solvent. The extract can also be prepared with varying degrees of purity using various concentrations and percentages of the butanol solvent and other solvents relatively similar in chemical composition to butanol.

vi. Stage 6—Further purification and separation of BuSY

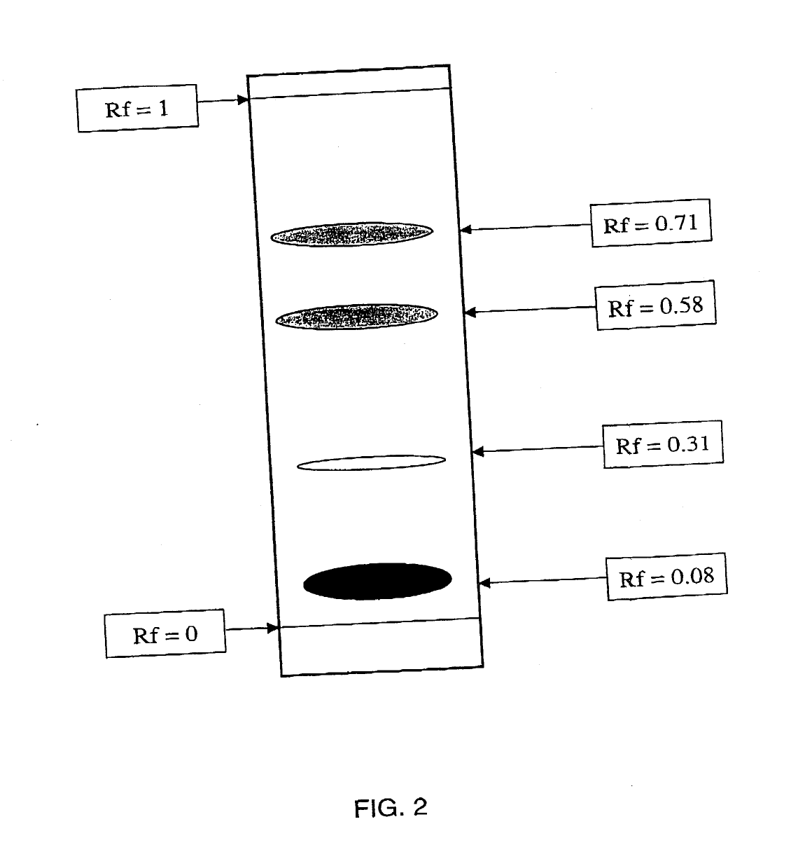

1. BuSY was further separated by TLC (Thin Layer Chromatography) method. The adsorbent was 25 TLC aluminum sheets (20×20 cm, silica gel 60 F254, EM SCIENCE, Germany) and the solvent system was butanol:ethyl acetate:methanol:water (4:2:1:1). The samples (10 (1) were added to the TLC sheet at the location of Rf=0 in a small TLC tank (10 ml solvent). Once in the vertical position, the solvent solution should not cover the sample-loading location, i.e. Rf=0. It took about 20-30 minutes for the solvent solution migrating up the TLC sheet to reach the top level, i.e. Rf=1. Then, the loaded BuSY were separated on the TLC sheet. Under UV 254 nm light, four main bands were found. The Rf values of the four bands were: 0.08, 0.31, 0.58 and 0.71, respectively (see FIG. 2). Correspondingly, these four fractions were named BuSY608, BuSY631, BuSY658, and BuSY671. This TLC purification technique demonstrates how many bands could be obtained and where they were located. Other higher throughput purification techniques, particularly column chromatography techniques, may be used to purify and isolate the BuSY extracts of this invention.

2. According to the indication of TLC, BuSY was separated by Column Chromatography method. Fixed phase was 10-40 (silica gel H (Sigma, USA). Mobile phase was butanol:ethyl acetate:methanol:water (4:2:1:1). The length and diameter of the column were 60 cm and 2.5 cm, respectively. First, the silica gel powder was added from the top of the column with a funnel to form the fixed phase that was compacted to a height of 45 cm. Then, 2 g of BuSY powder extract was added on top of the fixed phase. This BuSY layer was sandwiched by adding another 1 cm high silica gel powder. At last, the mobile phase was added on the top of BuSY sandwich. With this Column Chromatography method, the position of the BuSY powder layer was designated as Rf=0 and the bottom of the vertical column was the position Rf=1. The solvent (mobile phase) needed about 20-22 hrs to run from position Rf=0 to position Rf=1.

3. After column chromatography, the column was divided as four parts by calculating the Rf values obtained from TLC (FIG. 2). Each part of the silica gel with the separated BuSY was taken out from the column and soaked with 100 ml methanol in a beaker. Each fraction of the separated columns was soaked for 3-5 times, until no colour was seen in the methanol. The yield of BuSY608 separated from raw BuSY powder was 35.00%. The yield of BuSY608 from total methanol extract was 14.20%×35.00%=4.97%. The yield of BuSY608 from raw powder of Herba Cynomorii was 21.78%×14.20%×35.00%=1.08%.

4. After filtration with the 202 filter paper (Reeve Angel, USA), the filtrate was evaporated by an evaporator at the temperature of 45-80° C. and the separated powder was obtained at the bottom of the evaporator flask.

Other protocols with butanol for extracting from Cynomorium, most preferably Cynomorium songaricum Rupr. and the derived extracts with the function of treating erectile dysfunction as well as enhancing male sexual functions may be similarly developed within the present invention.

### EXAMPLE 2

Determining Relaxation of Isolated Rat Corpus Cavernosum Tissues by BuSY

This example demonstrates the relaxant effect on rat penile tissues of the extracts prepared from the herb Suo Yang in Example 1, i.e., BuSY and the further purified BuSY608.

i. Methods

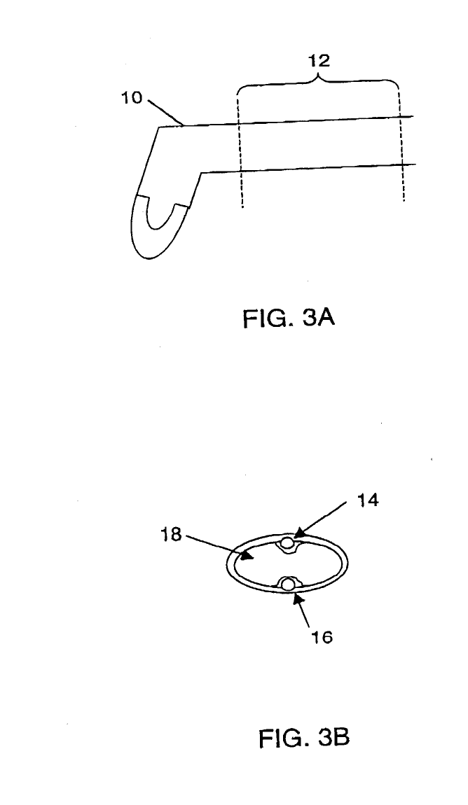

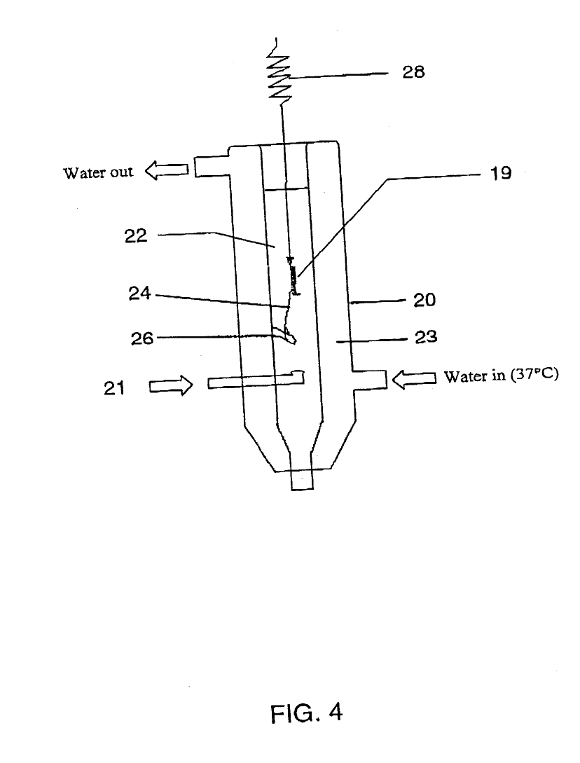

Experiments were performed on male, eight to twelve week old, 278.86±6.85 g Sprague-Dawley (SD) rats. Rats were housed in an animal care facility at the College of Medicine, University of Saskatchewan. Rats were anaesthetized with sodium pentobarbital (50 mg/kg) by intraperitoneal injection. The whole penis of one rat (shown at 10 in FIG. 3A) was gently dissected out and put into a Petri dish containing ice-cold Krebs' solution. The portion of the penis used for the experiments is shown generally at 12 in FIG. 3A. The penis was cleaned and the connective tissues were removed. The dorsal vein 14 and the urethra 16 were dissected and removed as shown in FIG. 3B. The section 12 was prepared for testing as a proximal section (10 mm in length) of the corpus cavernosum 18, which was cut longitudinally into two pieces and the size of each piece was about 1×1×10 mm. Each of the corpus cavernosum tissue strips 19, as prepared above, were mounted in an organ bath 20, as shown in FIG. 4, filled with 10 ml Krebs' bicarbonate solution 22 composed of (in mM): 115 NaCl, 5.4 KCl, 2.5 CaCl₂, 1.2 MgSO₂, 1.2 KH₂PO₄, 25 NaHCO₃, and 11 D-glucose, bubbled with 5% CO₂ in oxygen at inlet 21 and maintained at 37° C., with a circulating water bath 23. The pH of this bath solution 23 was adjusted to 7.2. The corpus cavemosum strips 19 were tied with silk threads 24 at each end with one end immobilized at 26 on the inner wall of the organ bath chamber 20, and the other hooked to a transducer 28 (FT 03, Grass Ins. Co., Quincy). The strips 19 were mechanically stretched to achieve a basal tension of approximately 0.5 g, and allowed to equilibrate for 1 hour before the experiment began. Strips 19 were contracted with 10 μM norepinephrine. After the plateau phase of contraction had been reached, the cumulative concentration-response relationships of BuSY and BuSY608 were tested. The concentration ladder was composed of (μg/ml): 2, 6, 20, 60, 200, 600, 2000, and 6000. As a control and comparison compound, sildenafil (VIAGRA®) was also used to test the penile tissue response (tissues prepared as above). Data acquisition and analysis were accomplished with a Biopac System (Biopac Systems, Inc., Golata) including the MP100 WS acquisition units, TCI 100 amplifiers, an AcKnowledge software (3.01), and an IBM computer.

ii. Results

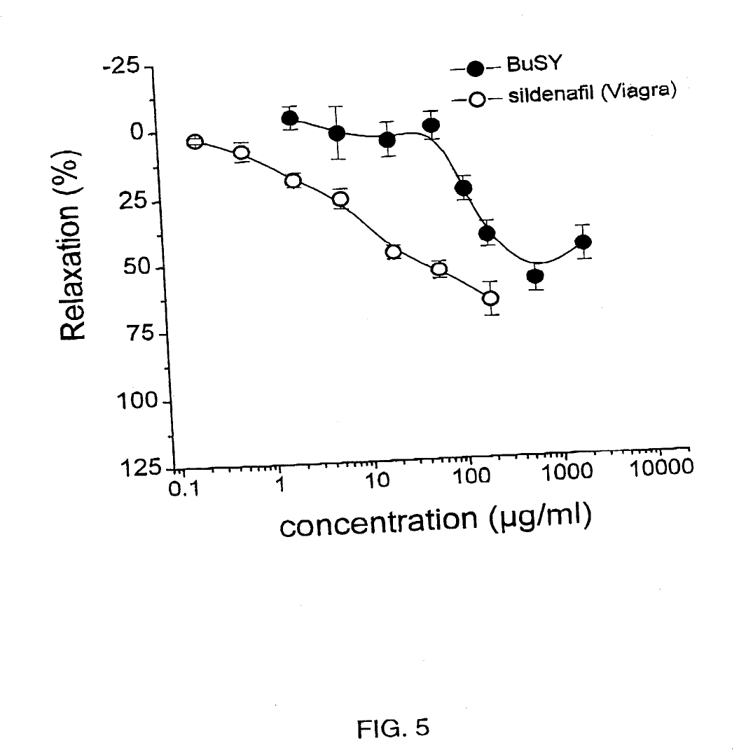

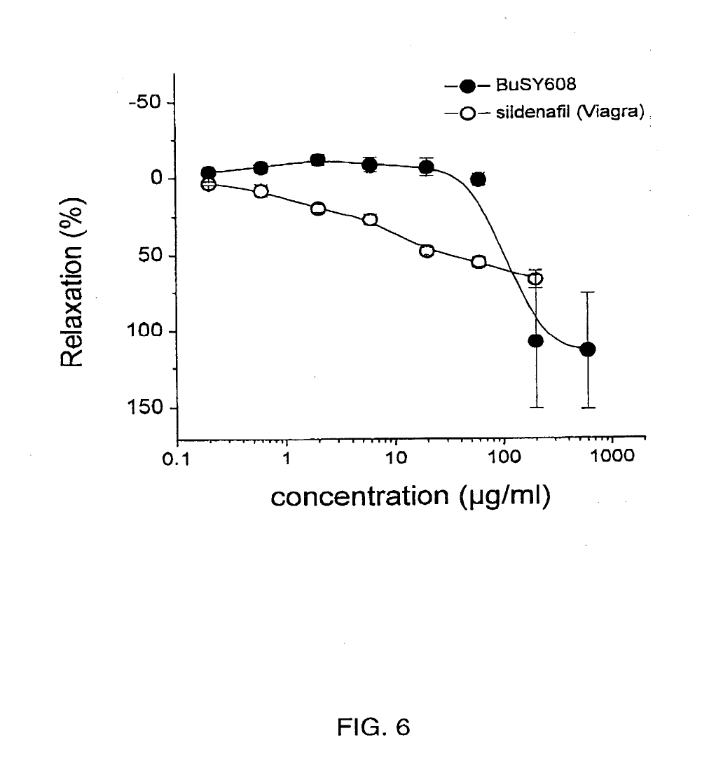

The effect of BuSY on the norepinephrine-precontracted rat corpus cavemosum strips (6 tissue samples) was tested. As shown in FIG. 5, BuSY relaxed corpus cavemosum tissue in a concentration-dependent manner. The relaxation induced by BuSY was initiated at 60 μg/ml and the EC50 was 129.1 (16.9 μg/ml. The maximal relaxation was 59.1±5.3% induced by 600 μg/ml of BuSY (n=6 representing the number of repeats of the experiment, i.e. 6 bath repeats for 6 tissue samples, with each tissue sample being tested over the 8 concentrations). Within the concentration range tested in the present study, sildenafil began to relax rat corpus cavemosum at 0.6 μg/ml and had an EC50 of 11.4 (4.6 μg/ml. The maximal relaxation was 66.7±6.1% induced by 200 μg/ml of sildenafil (n=6, FIG. 4). These results demonstrate that BuSY has a strong relaxant effect on norepinephrine-precontracted rat corpus cavemosum tissue comparable to sildenafil. Since the concentration of BuSY used in this study was based on the butanol fraction of Suo Yang of Example 1, but that of sildenafil was based on the mass of pure compound dissolved, further purification of BuSY is expected to generate more potent product in relaxing penile tissue than sildenafil. The relaxing effect of BuSY608, BuSY631, BuSY658, and BuSY671, the four components purified from BuSY using TLC or LC was also examined. Among these four components, BuSY608 has the best relaxing effect. As shown in FIG. 6, BuSY608 achieved a maximal relaxation of 113.9 (3.7% of the precontracted penile tissues at 600 μg/ml). This component has obvious stronger relaxing effect (EC50, 104.4(9.5 μg/ml) than BuSY, its parent extract, and is more potent in relaxing penile tissues than sildenafil in terms of the maximum relaxation effect.

BuSY and BuSY608 potently relaxed the pre-contracted rat corpus cavemosum strips in vitro. This is a predictor of prolonged erection of the penis in vivo for humans and other host animals. The maximum relaxant effect of BuSY was similar to, and that of BuSY608 was stronger than, that of sildenafil at the concentration range tested. Therefore, BuSY and BuSY608 show utility as anti-impotence drugs and as products to enhance male sexual function.

References:

Beijing Medicinal College, 1980, Component Chemistry of Traditional Chinese Medicine, People's Hygiene Press, Beijing, China, p. 17.

Chen Qi, 1994, Methodology of Pharmacology Study for Traditional Chinese Medicine, People's Hygiene Press, Beijing, China, p. 1103-1105.

Jiang Jiwu, 1990, Latin-Chinese Search Handbook of Medicinal Plant Name, China Medicinal Science and Technology Press, Beijing, China, p. 409.

Jiangsu New Medicinal College, 1986, Great Dictionary of Traditional Chinese Medicine, Shanghai Science and Technology Press, Shanghai, China, p. 4976.

Ou Ming, 1992, Chinese-English Manual of Common-used in Traditional Chinese Medicine, Guangdong Science and Technology Press, Guangzhou, China, p. 731-732.

Xi Nianzhu, 1997, Pharmaceutics, People's Hygiene Press, Beijing, China, p. 19, 55, 164,257,290.

Fitzpatrick et al., Endothelium-Dependent Vasorelaxation Caused by Various Plant Extract, J. of Cardiovascular Pharmacology, 26: 90-95, Raven Press Ltd, New York.

Merck Index, Merck & Co., Rahway, N.J.;

Gilman et al., (eds) (1996) Goodman and Gilman's: The Pharmacological Bases of Therapeutics, 8^{th} Ed., Pergamon Press.

Physicians Desk Reference (1997 Edition);

U.S. Pharmacopeia National Formulary (1995) United States Pharmacopeial Convention Inc., Rockville, Md.

All publications mentioned in this specification are indicative of the level of skill in the art to which this invention pertains. To the extent they are consistent herewith, all publications mentioned in this specification are herein incorporated by reference to the same extent as if each individual publication was specifically and individually indicated to be incorporated by reference. No admission is made that any cited reference constitutes prior art.

Although the foregoing invention has been described in some detail by way of illustration and example, for purposes of clarity and understanding it will be understood that certain changes and modifications may be made without departing from the scope or spirit of the invention as defined by the following claims.

We claim:

1. An extract of Cynomorium obtained by solvent extraction from the stems of a plant of the family Cynomoriaceae.

2. The extract, of claim 1, wherein the plant is selected from the group consisting of Cynomorium songaricum Rupr. and Cynomorium coccineum L.

3. The extract of claim 2, wherein the plant is Cynomorium songaricum Rupr.

4. The extract of claim 3, wherein the extract is prepared from the stem of the plant.

5. The extract of claim 4, wherein the extract is obtained from an extraction solvent selected from the group consisting of one or more of butanol, methanol, ethanol, propanol, isobutyl alcohol, pentanol, isoamyl alcohol, hexanol, hexanediol, acetone, ethyl acetate, and water.

6. The extract of claim 4, wherein the extract is obtained from an extraction solvent which includes butanol.

7. The extract of claim 6, wherein the extract is purified.

8. A pharmaceutical composition comprising an extract of Cynomorium of claim 1, in admixture with one or more pharmaceutically acceptable carriers.

9. The pharmaceutical composition of claim 8 wherein the plant is selected from the group consisting of Cynomorium songaricum Rupr. and Cynomorium coccineum L.

10. The pharmaceutical composition of claim 9, wherein the plant is Cynomorium songaricum Rupr.

11. The pharmaceutical composition of claim 10, wherein the extract is prepared from the stem of the plant.

12. The pharmaceutical composition of claim 11, wherein the extract is obtained from an extraction solvent which includes butanol.

13. The pharmaceutical composition of claim 12, in a dosage form to deliver 0.001 to 30 g of the extract on a daily basis.

14. The pharmaceutical composition of claim 12, in a dosage form to deliver 0.5 to 20 g of the extract on a daily basis.

15. The pharmaceutical composition of claim 12, in a dosage form to deliver 2 to 15 g of the extract on a daily basis.

16. The pharmaceutical composition of claim 12, in a dosage form to deliver 5 to 10 g of the extract on a daily basis.

17. A method of treating erectile dysfunction comprising:
administering a therapeutically effective amount of an extract of Cynomorium to a host animal.

18. The method of claim 17, wherein the plant is selected from the group consisting of Cynomorium songaricum Rupr. and Cynomorium coccineum L.

19. The method of claim 17, wherein the plant is Cynomorium songaricum Rupr.

20. The method of claim 19, wherein the extract is prepared from the stem of the plant.

21. The method of claim 20, wherein the extract is obtained from an extraction solvent which includes butanol.

22. The method of claim 21, wherein the extract is purified.

23. The method of claim 22, wherein the extract is in a dosage form to deliver 0.001 to 30 g of the extract on a daily basis.

24. The method of claim 22, wherein the extract is in a dosage form to deliver 0.5 to 20 g of the extract on a daily basis.

25. The method of claim 22, wherein the extract is in a dosage form to deliver 2 to 15 g of the extract on a daily basis.

26. The method of claim 22, wherein the extract is in a dosage form to deliver 5 to 10 g of the extract on a daily basis.

27. A method of preparing an extract of Cynomorium, comprising:
contacting a powder or pulp obtained from the plant or plant parts of the family Cynomoriaceae with one or more organic or aqueous extraction solvents to remove an extract; and
isolating the extract with positive activity for alleviating or preventing symptoms of erectile dysfunction.

28. The method of claim 27, wherein the plant is selected from the group consisting of Cynomorium songaricum Rupr. and Cynomorium coccineum L.

29. The method of claim 27, wherein the plant is Cynomorium songaricum Rupr.

30. The method of claim 29, wherein the extract is prepared from the stem of the plant.

31. The method of claim 30, wherein the extract is obtained from an extraction solvent selected from the group consisting of one or more of butanol, methanol, ethanol, propanol, isobutyl alcohol, pentanol, isoamyl alcohol, hexanol, hexanediol, acetone, ethyl acetate, and water.

32. The method of claim 30, wherein the extract is obtained from an extraction solvent which includes butanol.

33. The method of claim 32, wherein the extract is purified.

34. The method of claim 33, wherein the extract is prepared by:
contacting a powder or pulp derived from the plant with a first solvent which includes methanol, followed by filtration and solvent removal to obtain a first extract;
contacting the first extract with water and hexane and recovering a first water fraction;
contacting the first water fraction with chloroform and recovering a second water fraction;
contacting the second water fraction with ethyl acetate and recovering a third water fraction; and
contacting the third water fraction with butanol, recovering a butanol fraction and removing the butanol solvent to obtain an extract.

35. The method of claim 34, wherein the extract is purified by column chromatography.

36. The method of claim 35, wherein the extract is purified by thin layer chromatography and or liquid chromatography.
